(19) United States
(12) Patent Application Publication
(10) Pub. No.: US 2023/0173123 A1
(43) Pub. Date: Jun. 8, 2023

(54) LOCALIZED CLEANING USING MOBILE AIR CLEANER

(71) Applicant: Carrier Corporation, Palm Beach Gardens, FL (US)

(72) Inventors: Michael J. Birnkrant, Manlius, NY (US); Alissa D. Doose, Denver, CO (US); Chrystal Jolliffe, London, OH (US)

(21) Appl. No.: 18/072,376

(22) Filed: Nov. 30, 2022

Related U.S. Application Data
(60) Provisional application No. 63/285,825, filed on Dec. 3, 2021.

(51) Int. Cl. A61L 9/00

(52) U.S. Cl. CPC A61L 9/00; A61L 2209/111; A61L 2209/14; A61L 2209/15

(57) ABSTRACT

A cleaning device includes a body, an air flow path extending through the body, an air filtration system arranged within the air flow path, and a propulsion system to maneuver the body through an area to be cleaned.

### CROSS-REFERENCE TO RELATED APPLICATIONS

This application claims the benefit of U.S. Provisional Application No. 63/285,825 filed Dec. 3, 2021, the disclosure of which is incorporated herein by reference in its entirety.

### BACKGROUND

Embodiments of the present disclosure relate to the art of air purification, and more particularly, to an autonomous air purification system.

A reduced air quality as a result of the presence of airborne pollutants can have a negative impact on a person's health when the concentration of the pollutants exceeds the standard. Household air purification devices or filters are available. However, these tend to be standalone units that remain at a single location within the home. Accordingly, to achieve a desired air quality throughout a home or the main living areas of the home, several air purification devices are required.

### BRIEF DESCRIPTION

According to an embodiment, a cleaning device includes a body, an air flow path extending through the body, an air filtration system arranged within the air flow path, and a propulsion system to maneuver the body through an area to be cleaned.

In addition to one or more of the features described above, or as an alternative to any of the foregoing embodiments the air filtration system further comprises at least one filter, an air movement mechanism operable to move a flow of air through the at least one filter, and a motor operably coupled to the air movement mechanism.

In addition to one or more of the features described above, or as an alternative to any of the foregoing embodiments the propulsion system further comprises a plurality of wheels configured to support the body on a floor and a drive motor operably coupled to at least one of the plurality of wheels.

In addition to one or more of the features described above, or as an alternative to any of the foregoing embodiments the motor is the drive motor.

In addition to one or more of the features described above, or as an alternative to any of the foregoing embodiments the propulsion system further comprises at least one rotor mounted to the body and a drive motor operably coupled to the at least one rotor.

In addition to one or more of the features described above, or as an alternative to any of the foregoing embodiments the motor is the drive motor.

In addition to one or more of the features described above, or as an alternative to any of the foregoing embodiments the air flow path further comprises an air inlet arranged at a first portion of the body and an air outlet at a second portion of the body.

In addition to one or more of the features described above, or as an alternative to any of the foregoing embodiments the body further comprises a front, a back, a top, and a bottom, the front being the first portion and the back being the second portion.

In addition to one or more of the features described above, or as an alternative to any of the foregoing embodiments further comprising an air quality sensor configured to detect an air quality and a controller configured to operate the air filtration system when the air quality is less than a predetermined threshold.

In addition to one or more of the features described above, or as an alternative to any of the foregoing embodiments further comprising at least one of a position sensor and an obstacle detection sensor, wherein the controller is configured to operate the propulsion system in response to the at least one of the position sensor and the obstacle detection sensor.

In addition to one or more of the features described above, or as an alternative to any of the foregoing embodiments the controller is configured to operate the propulsion system when the air quality is greater than or equal to the predetermined threshold.

In addition to one or more of the features described above, or as an alternative to any of the foregoing embodiments the propulsion system is operable to move the body autonomously.

According to an embodiment, a method of performing an air cleaning operation includes autonomously moving a cleaning device throughout a space, detecting an air quality of an air surrounding the cleaning device, and filtering the air surrounding the cleaning device until the air quality is at least equal to a predetermined threshold.

In addition to one or more of the features described above, or as an alternative to any of the foregoing embodiments further comprising stopping movement of the cleaning device in response to the air quality of the air surrounding the cleaning device being below the predetermined threshold.

In addition to one or more of the features described above, or as an alternative to any of the foregoing embodiments filtering the air surrounding the cleaning device further comprises drawing the air through at least one filter arranged within the interior of the cleaning device via an air movement mechanism.

In addition to one or more of the features described above, or as an alternative to any of the foregoing embodiments further comprising autonomously moving the cleaning device in response to detecting the air quality of the air surrounding the cleaning device to be equal to or greater than the predetermined threshold.

In addition to one or more of the features described above, or as an alternative to any of the foregoing embodiments autonomously moving the cleaning device throughout the space is controlled remotely via a portable device.

In addition to one or more of the features described above, or as an alternative to any of the foregoing embodiments further comprising indicating via a display a status of one or more operational parameters of the cleaning device.

In addition to one or more of the features described above, or as an alternative to any of the foregoing embodiments scheduling of the air cleaning operation is controlled remotely via a portable device.

### BRIEF DESCRIPTION OF THE DRAWINGS

The following descriptions should not be considered limiting in any way. With reference to the accompanying drawings, like elements are numbered alike:

FIG. 1 is a schematic diagram of an exemplary autonomous cleaning device according to an embodiment;

FIG. 2 is a perspective view of an exemplary autonomous cleaning device according to an embodiment;

FIG. 3 is a side view of the autonomous cleaning device of FIG. 2 according to an embodiment;

FIG. 4 is a side view of another exemplary autonomous cleaning device according to an embodiment;

FIG. 5 is a schematic diagram of another exemplary autonomous cleaning device according to an embodiment;

FIG. 6 is a front view of an exemplary user interface of the cleaning device according to an embodiment;

FIG. 7 is a front view of a display of an exemplary user interface of the cleaning device according to an embodiment; and

FIG. 8 is a schematic diagram of an exemplary control system of the cleaning device according to an embodiment.

### DETAILED DESCRIPTION

A detailed description of one or more embodiments of the disclosed apparatus and method are presented herein by way of exemplification and not limitation with reference to the Figures.

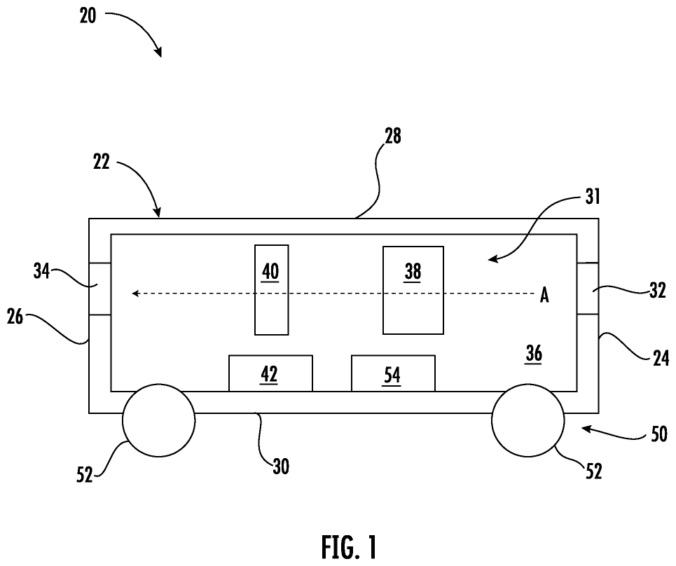

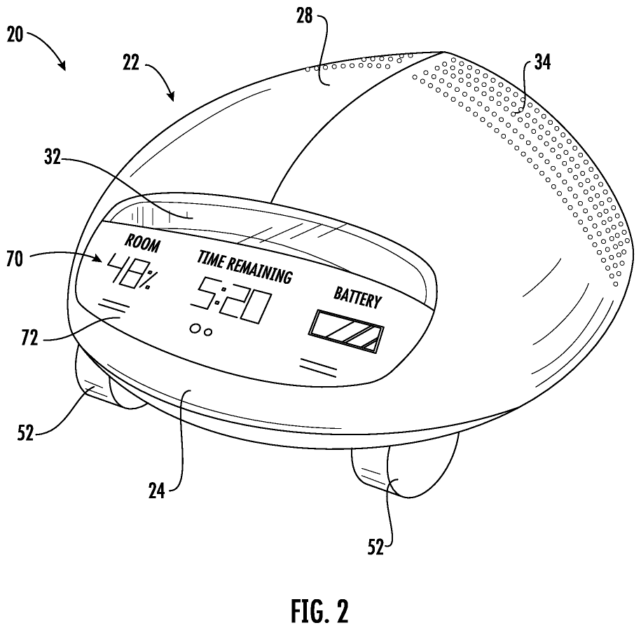

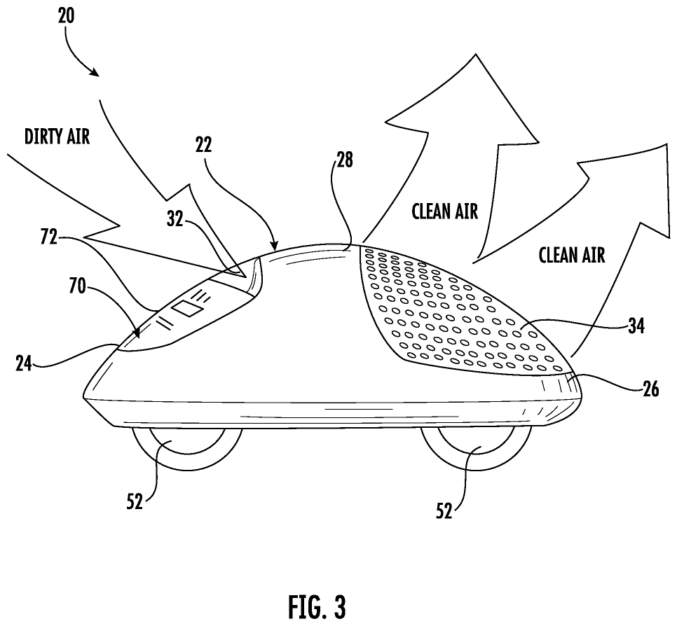

With reference now to FIG. 1, an exemplary cleaning device 20 is illustrated. As shown, the cleaning device 20 includes a body 22 having a front or forward portion 24, a back or rear portion 26, a top 28 and a bottom 30. In the non-limiting embodiment of FIG. 1, the body 22 has a generally rectangular configuration. However, in the embodiments of FIGS. 2 and 3, the body 22 is shown having a semi-circular configuration and a circular configuration, respectively. It should be understood that the embodiments illustrated and described herein are intended as an example only and that a body 22 having another suitable configuration is within the scope of the disclosure.

An air filtration system 31 is configured to clean a flow of air A as it moves along an air flow path extending between an air inlet 32 and an air outlet 34 of the cleaning device 20. The air inlet 32 is arranged at a first portion of the body 22, such as at the front 24 of the body 22 for example, and the air outlet 34 is arranged at a second portion of the body 22, such as at the rear portion of the body 22 for example. It should be understood that that air inlet 32 and the air outlet 34 may be arranged at any surface of the body 22, respectively. In an embodiment, the air inlet 32 and the air outlet 34 are arranged in different planes, such as at different sides of the body 22 to prevent the clean air exhausted via the air outlet 34 from being recirculated directly to the air inlet 32. Each of the air inlet 32 and the air outlet 34 is arranged in fluid communication with an interior 36 of the body 22 such that the air flow path extends through the interior 36 of the body 22.

FIG. 1, the air inlet 32 of the cleaning device 20 utilizes the display 70 to increases the environmental robustness of the unit. The overhang between display 70 and air inlet 32 helps reduce liquids from entering the device but still directing the air towards an internal fan. The circular air intake also reduces noise by evenly loading the internal fan on the interior 36 of the body 22.

In an embodiment, the air filtration system 31 includes at least one filter 38 arranged within the interior 36 of the body 22. The one or more filters 38 are configured to remove bacteria, viruses and particulate matter from the air provided thereto. In an embodiment, the at least one filter is a carbon filter or a HEPA-type filter. In an embodiment, the filter 38 includes a photocatalytic material or coating. In such embodiments, the filter 38 may be decontaminated or cleaned by emitting a light, such as from a light source (not shown), having a desired wavelength over the filter 38. However, any suitable type of filter 38 or combination of filters is within the scope of the disclosure.

The air filtration system 31 additionally includes an air movement mechanism 40, such as a fan for example, arranged within the air flow path. Operation of the air movement mechanism 40 is configured to move air along the air flow path, such as by drawing air into the air inlet 32, through the filter 38, and exhausting the filtered air via the air outlet 34. Although the air movement mechanism 40 is illustrated as being arranged within the interior 36 of the body 22, downstream from the filter 38 relative to the air flow (i.e. in a draw-through configuration), embodiments where the air movement mechanism 40 is arranged at an exterior of the body 22 and/or where the air movement mechanism 40 is arranged upstream from a filter 38 (i.e. in a blow-through configuration) are also contemplated herein.

In an embodiment, the air movement mechanism 40 is driven by a motor 42 positioned within the interior of the body 22. The motor 42 may be coaxial with the air movement mechanism 40, such that the motor 42 is generally within the air flow path. In another embodiment, best shown in FIG. 1, the motor 42 is mounted separately from the air movement mechanism 40 such that the motor 42 is generally remote from the air flow path.

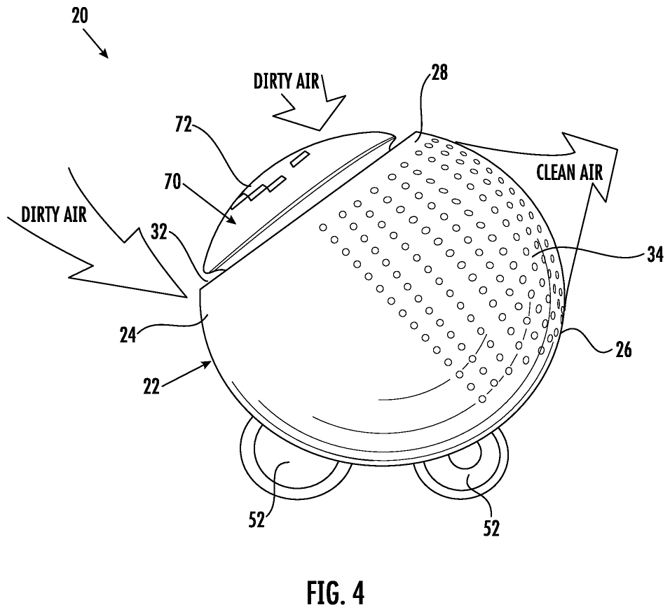

The cleaning device 20 additionally includes a propulsion system 50 configured to move the cleaning device 20 throughout a space. In the non-limiting embodiment of FIGS. 1-4, the propulsion system 50 includes a plurality of wheels 52 that rotatably support the body 22 of the cleaning device 20. The wheels 52 may be mounted about one or more axles. Accordingly, each wheel 52 may be mounted about a separate axle, or in other embodiments, two or more wheels 52 may be mounted to the same axle, such as extending between opposite sides of the body 22 for example. In other embodiments, one or more of the wheels 52 may be caster wheels, or alternatively, mecanum wheels.

At least one drive motor 54 is coupled to one or more of the plurality of wheels 52 to drive the cleaning device 20 across a floor surface by rotating the wheels 52. Accordingly, in an embodiment, only one of the wheels 52 is driven by the drive motor 54 and the remainder of the plurality of wheels 52 are indirectly driven. In other embodiments, more than one, and in some embodiments each of the plurality of wheels is driven by the one or more drive motors 54. Although a single drive motor 54 is illustrated in the FIGS., embodiments where a plurality of drive motors are operably coupled to a single wheel, or alternatively to a plurality of wheels 52 is also within the scope of the disclosure. It should be understood that the same motor 42 operable to drive the air movement mechanism 40 may be used to drive one or more of the wheels 52 of the propulsion system 50.

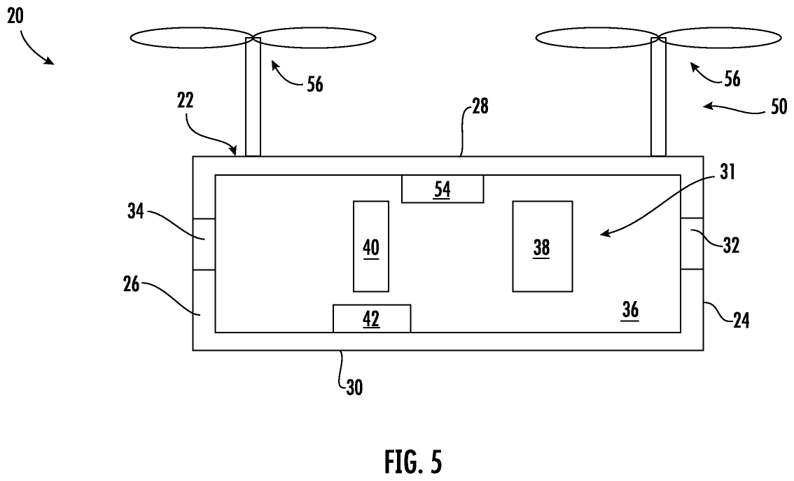

With reference now to FIG. 5, in another embodiment, the propulsion system 50 includes one or more rotors 56 extending from or integrally formed with the body 22 of the cleaning device 20. At least one drive motor 54 is coupled to the at least one rotor 56 to move, for example fly or maneuver the cleaning device 20 through the air within the space. A single drive motor or multiple drive motors 54 may be operably coupled to the plurality of rotors 56. Alternatively, the propulsion system 50 may include a distinct drive motor 54 operably coupled to and configured to drive a respective rotor of the plurality of rotors 56. As previously suggested, the motor 42 associated with the air movement mechanism 40 may also be operably coupled and therefore used to drive one or more of the rotors 56 of the propulsion system 50. In embodiments where the same motor 42/54 is incorporated into both the air filtration system 31 and the propulsion system 50, only one of the air filtration system 31 and the propulsion system 50 may be operable at a time. Accordingly, the cleaning device 20 may be operable to either filter the air, or move the cleaning device 20 at any given time. However, embodiments where the cleaning device 20 is simultaneously movable and operable to clean the air are also contemplated herein.

It should be understood that the cleaning device 20 may include another cleaning system operable to perform a different type cleaning operation. For example, the cleaning device 20 may also be configured to vacuum or collect dirt and debris, such as during movement to a location with a reduced air quality. Accordingly, in an embodiment, the cleaning device 20 is similar to an existing robotic vacuum having an air filtration system 31 incorporated therein to perform air purification.

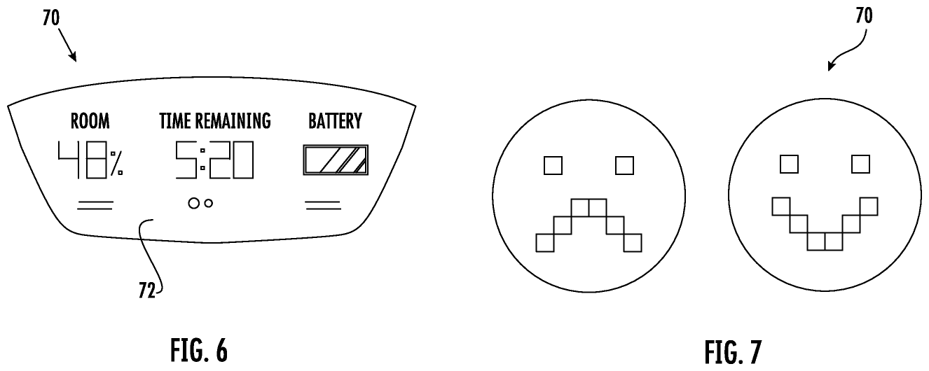

The cleaning device 20 includes one or more batteries 60 (shown in FIG. 6) sufficient to provide the power necessary to operate the cleaning device 20, such as the at least one motor 42 and/or drive motor 54 for an extended period of time. In an embodiment, the cleaning device 20 is connectable with a docking station to charge or recharge the one or more batteries 60 within the cleaning device 20. Further, when the cleaning device 20 is connected to the docking station, the docking station may be able to perform a self- cleaning operation to remove particulate matter or debris that has accumulated on the filter. Such a self-cleaning operation may simply include drawing matter away from the filter 38, such as via a movement mechanism for example, or alternatively, may include sterilizing the filter by emitting a light having a desired wavelength, such as an ultraviolet light for example, over the filter 38.

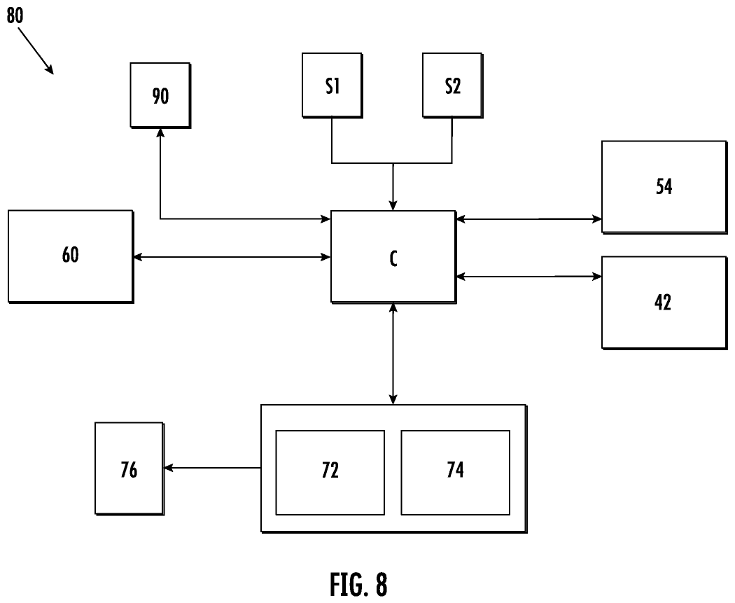

The cleaning device 20 additionally includes a control panel or user interface 70 positioned adjacent one or more sides of the body 22. In an embodiment, at least a portion of the air inlet 32 is arranged adjacent to or surrounds part of the user interface 70. An example of such a user interface 70 is shown in FIGS. 6 and 7. As shown, the user interface 70 may include a display 72 configured to indicate one or more of an air quality surrounding the cleaning device 20, the battery life, and the elapsed time or time remaining associated with a cleaning cycle. The air quality information may be provided as a rating, such as based on a percentage of pollutants detected within the air, or alternatively, may be provided simply as either good or bad based on the measured air quality relative to the predetermined threshold. The simple indication of good or bad may be shown via any suitable method, such as via a green or red light, or via a happy face or sad face (FIG. 7) for example.

The user interface 70 may additionally include one or more inputs 74 (illustrated schematically in FIG. 8) associated with operation of the propulsion system 50 and/or the air filtration system 31 of the cleaning device 20. For example, an input 74 may be associated with selecting a mode of operation of the cleaning device 20. In an embodiment, at least one input 74 on the user interface 70 is an on/off button which allows the user to activate or deactivate the user interface 70. Alternatively, or in addition, the at least one input 74 is a start button intended to initiate operation of the cleaning device 20. A sound generation mechanism 76, such as a speaker for example, may be mounted within the interior of the body 22 and connected to the user interface 70. The sound generation mechanism 76 may be operable to generate a noise in response to detection of a condition, to indicate a status of the battery 60, or to indicate that a cleaning cycle has been initiated or is complete for example.

A control system 80 of the cleaning device 20 includes a controller or processor C for controlling operation of the air filtration system 31 and the propulsion system 50, and in some embodiments for executing stored sequences of air filtration and/or propulsion. The controller C is operably coupled to the user interface 70 and to the motor 42 and/or the drive motor 54. In an embodiment, the control system 80 includes at least one of a position sensor and an obstacle detection sensor (illustrated schematically at Si) associated with the propulsion system 50. Examples of such sensors Si include mechanical drop sensors and light-based proximity sensors for example. In such embodiments, the controller C may use the sensor Si to detect an obstacle prior to a collision therewith and maneuver the cleaning device 20 away from the obstacle. Alternatively, or in addition, the controller C may be configured to control the propulsion system 50 to maneuver the cleaning device 20 from an obstacle in response to collision with the obstacle.

In addition, in an embodiment, the control system 80 includes one or more sensors S2 associated with the air filtration system 31. For example, the cleaning device 20 includes a sensor S2 configured to monitor an air quality of the atmosphere surrounding the exterior of the cleaning device 20. Examples of such an air quality sensor S2 include a pollution sensor such as for measuring ozone, particulate matter, carbon monoxide, sulfur dioxide, and nitrous oxide. Such an air quality sensor S2 may be a photometer which detects the presence of pollutants based on the scattered light reflected back to the sensor. However, any suitable air quality sensor is within the scope of the disclosure.

The cleaning device 20 may be configured to execute a variety of navigational behaviors. Examples of navigational behaviors include movement in a straight line or movement in any suitable pattern. The navigational behavior may be selected based on a mode of operation of the cleaning device 20. The cleaning device 20 may be configured to move through the area or space to be cleaned in any suitable manner. In an embodiment, the area or space to be cleaned is a single room. In other embodiments, the area or space to be cleaned by the cleaning device 20 includes a plurality of rooms connected to one another.

In an embodiment, the cleaning device 20 is configured to monitor an air quality of the atmosphere surrounding the cleaning device 20, such as during the movement of the cleaning device 20 via the propulsion system. The cleaning device 20 may be configured to move towards a location having a lower or reduced air quality. Specifically, in combination with its movement, the cleaning device 20 may be configured to use the at least one air quality sensor to determine a location of one or more pollutants within a room. Upon detecting a location with an air quality below the allowable threshold, operation of the propulsion system 50 may stop or cease and operation of the air filtration system 31 may be initiated to actively clean or purify the air directly adjacent to or surrounding the pollutant(s). Examples of pollutants include, but are not limited to people, pets, and cooking devices that generate airborne particles during operation. In an embodiment, once operation of the air filtration system 31 is initiated, the cleaning device 20 is configured to remain at that location until the air quality of the air surrounding the cleaning device exceeds the predetermined threshold. Once the air quality is equal to or exceeds the predetermined threshold, operation of the air filtration system 31 may cease, and the propulsion system 50 may be energized. The cleaning device 20 may be configured to travel from one room to the next room once the air quality within a predefined area or room is above a predetermined threshold.

In an embodiment, the cleaning device 20 is controllable remotely, such as via a portable device, illustrated schematically at 90. Examples of a portable device 90 include, but are not limited to, a computer, a tablet, a personal digital assistant (PDA), a smartphone, a cellular device with SMS capabilities, or any other device configured to generate a signal. The portable device 90 may communicate with the controller C via any suitable type of communication including, but not limited to, Bluetooth, a local or public data network, radio frequency, a cellular network, near field communication, or any other wireless or wired network. In an embodiment, an application or “app” associated with the portable device 90 is used to establish communication with the controller C, such as to initiate operation thereof, or command movement of the cleaning device 20 to a specific location.

In an embodiment, the app is operable to set a schedule for operation of the cleaning device 20. The customization of the schedule available via the app may include selecting a time of day to perform an air purifying or air cleaning operation, the frequency at which a cleaning operation is performed, specific areas or rooms to be cleaned, and or the desired air quality level to be achieved associated with each room. The app may also be used to designate areas that should not be included in the area to be cleaned. Further, the app may be operable to convey to the user one or more status or operational parameters of the cleaning device, such as the status and operational parameters that are visible via the display 72 of the cleaning device 20. In addition, in an embodiment, the app is operable to convey the air quality levels before and after a cleaning operation is performed, thereby indicating the improvement in the air quality achieved in each area that was cleaned.

The cleaning device 20 described herein provides a single whole-home air purification system capable of moving autonomously throughout a designated area. Further, the cleaning device 20 is capable of providing customized air purification based on the layout and air quality demands of the area to be purified, and the scheduling needs of the users.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A cleaning device comprising:
a body;
an air flow path extending through the body;
an air filtration system arranged within the air flow path; and
a propulsion system to maneuver the body through an area to be cleaned.

2. The cleaning device of claim 1, wherein the air filtration system further comprises:
at least one filter;
an air movement mechanism operable to move a flow of air through the at least one filter; and
a motor operably coupled to the air movement mechanism.

3. The cleaning device of claim 2, wherein the propulsion system further comprises:
a plurality of wheels configured to support the body on a floor; and
a drive motor operably coupled to at least one of the plurality of wheels.

4. The cleaning device of claim 3, wherein the motor is the drive motor.

5. The cleaning device of claim 2, wherein the propulsion system further comprises:
at least one rotor mounted to the body; and
a drive motor operably coupled to the at least one rotor.

6. The cleaning device of claim 5, wherein the motor is the drive motor.

7. The cleaning device of claim 1, wherein the air flow path further comprises an air inlet arranged at a first portion of the body and an air outlet at a second portion of the body.

8. The cleaning device of claim 7, wherein the body further comprises a front, a back, a top, and a bottom, the front being the first portion and the back being the second portion.

9. The cleaning device of claim 1, further comprising:
an air quality sensor configured to detect an air quality; and
a controller configured to operate the air filtration system when the air quality is less than a predetermined threshold.

10. The cleaning device of claim 9, further comprising at least one of a position sensor and an obstacle detection sensor, wherein the controller is configured to operate the propulsion system in response to the at least one of the position sensor and the obstacle detection sensor.

11. The cleaning device of claim 10, wherein the controller is configured to operate the propulsion system when the air quality is greater than or equal to the predetermined threshold.

12. The cleaning device of claim 9, wherein the propulsion system is operable to move the body autonomously.

13. A method of performing an air cleaning operation comprising:
autonomously moving a cleaning device throughout a space;
detecting an air quality of an air surrounding the cleaning device; and
filtering the air surrounding the cleaning device until the air quality is at least equal to a predetermined threshold.

14. The method of claim 13, further comprising stopping movement of the cleaning device in response to the air quality of the air surrounding the cleaning device being below the predetermined threshold.

15. The method of claim 13, wherein filtering the air surrounding the cleaning device further comprises drawing the air through at least one filter arranged within the interior of the cleaning device via an air movement mechanism.

16. The method of claim 13, further comprising autonomously moving the cleaning device in response to detecting the air quality of the air surrounding the cleaning device to be equal to or greater than the predetermined threshold.

17. The method of claim 16, wherein autonomously moving the cleaning device throughout the space is controlled remotely via a portable device.

18. The method of claim 13, further comprising indicating via a display a status of one or more operational parameters of the cleaning device.

19. The method of claim 13, wherein scheduling of the air cleaning operation is controlled remotely via a portable device.
